(19) United States
(12) Patent Application Publication
(10) Pub. No.: US 2011/0070639 A1
(43) Pub. Date: Mar. 24, 2011

(54) METHOD OF DESIGNING HYDRODYNAMIC CAVITATION REACTORS FOR PROCESS INTENSIFICATION

(75) Inventors: Aniruddha Bhalchandra Pandit, Maharashtra (IN); Anjan Charan Mukherjee, Maharashtra (IN); Gopal Rameschandra Kasat, Maharashtra (IN); Amit Vinod Mahulkar, Maharashtra (IN)

(73) Assignee: HYCA Technologies Pvt. Ltd., Mumbai (IN)

(21) Appl. No.: 12/992,038

(22) PCT Filed: May 13, 2009
(86) PCT No.: PCT/IN09/00280

§ 371 Date: Nov. 10, 2010

(30) Foreign Application Priority Data
May 15, 2008 (IN) 1045/MUM/2008

(51) Int. Cl. B01J 19/00; C12M 1/33; G06F 17/50; G06F 17/10

(52) U.S. Cl. 435/306.1; 422/127; 703/1

(57) ABSTRACT

The present invention describes an apparatus of Hydrodynamic cavitation, to be used as reactors to achieve tangible effect by producing tailored active cavities either transient or steady or both, in aqueous and non-aqueous media for intensification of the physical and chemical processes in homogenous and heterogeneous systems. An apparatus comprises of a cavity generator, cavity diverter and turbulence manipulator wherein the cavity generator/cavity diverter is a flow modulator of various shapes and sizes. A regime map of cavitation and a method to generate it, is presented to achieve the desired type of cavitation, required for specific targeted process intensification and then reactors are designed to achieve the predetermined process intensification. Regime map relates the maximum fluid velocity in cavity generator with the cavitation number, active and specific type of cavity fraction for several geometric designs of apparatus.

### FIELD OF INVENTION

This invention relates to hydrodynamic cavitation reactors to achieve tailored cavitating conditions in aqueous and non-aqueous media, for intensification of the physical and chemical processes and a method for designing such reactors.

### BACKGROUND AND PRIOR ART

‘Process Intensification” involves providing energy efficient, and environmentally safe processes using compact production equipment for the production of quality products, minimizing waste generation, resulting in substantial cost reduction thereby enhancing the sustainability of advanced technologies.

Cavitation has gained importance in recent times as it provides a means of generating local conditions of high temperatures (˜14 000 K) and pressures (˜10 000 atm) at nearly ambient bulk processing conditions. The collapse or implosion of the formed cavities results in short-lived, localized hot-spots in cold liquid which can be effectively exploited to carry out physico-chemical processes including intensification of the chemical reactions, acoustic streaming in the reactor and enhancing the rates of transport processes.

Generally, cavitation is classified into four types based on the mode of generation,

Acoustic cavitation—produced by passage of ultrasound through the fluid.
Hydrodynamic cavitation—produced by creating pressure variations in the flowing fluid.
   Optic Cavitation—produced by passing the photons of high intensity light through the liquid.
   Particle cavitation—produced by bombardments of high energy particles such as proton or neutron in the liquid.

Among the various modes of generating cavitation given above, hydrodynamic cavitation can be applied for the intensification of the physico-chemical processes to large scale liquid volumes on industrial scale.

Senthilkumar et al. (2000) [SenthilKumar, P., Sivakumar, M. & Pandit, A. B. Experimental quantification of chemical effects of hydrodynamic cavitation. Chemical Engineering Science, 55, 1633-1639, 2000.] have shown that, hydrodynamic cavitation can be generated by the passage of the liquid through a constriction such as throttling valve, orifice plate, venturi etc. Gogate et al. (2006) [Gogate, P. R. & Pandit, A. B. A review and assessment of hydrodynamic cavitation as a technology for the future. Ultrasonic Sonochemistry, 12, 21-27, 2005.] have discussed the use of hydrodynamic cavitation for the intensification of the chemical processes such as oxidation of toluene, (o-/p-/m)-xylenes, mesitylene, (o-/m)-nitrotoluenes and (o-/p)-chlorotoluenes; transesterification of vegetable oils by using alcohols has been discussed by Kelkar & Pandit (2005) [Kelkar, M. A. & Pandit, A. B. Cavitationally Induced Chemical Transformations. M. Chem. Engg. Thesis, University of Mumbai, 2005]; esterification of fatty acids using alcohols has bee discussed by Kelkar et al. (2008) [Kelkar, M. A., Gogate, P. R. & Pandit, A. B. Intensification of esterification of acids for synthesis of biodiesel using acoustic and hydrodynamic cavitation. Ultrasonic Sonochemistry, 15, 188-194, 2008]. Similarly hydrodynamic cavitation has been applied to microbial disruption for the disinfection of potable water (Jyoti & Pandit, 2002) [Jyoti, K. K. & Pandit, A. B. Studies in water disinfection techniques. Ph.D. (Tech) Thesis, University of Mumbai, 2002]; Cell disruption for the release of intracellular enzymes [Balasundaram, B. & Harrison, S. T. L. Study of Physical and Biological Factors Involved in the Disruption of E. Coli by Hydrodynamic Cavitation]; Emulsification [Gaikwad, S. G. & Pandit, A. B. Application of Ultrasound in Heterogeneous Systems. Ph.D. (Tech) Thesis, University of Mumbai, 2007]; nano particle synthesis [Patil, M. N. & Pandit, A. B. Cavitation-A Novel Technique for making stable nano-suspensions. Ultrasonics Sonochemistry, 14, 519-530, 2007].

In hydrodynamic cavitation the intensity of the cavitation prevailing in the reactor is related to the global operating conditions through the cavitation number. Cavitation number can be mathematically represented as:

$${C}_{v} = \frac{{P}_{2} - {P}_{v}}{\frac{1}{2} {ρ}_{l} {v}_{o}^{2}} \qquad ( 1 )$$

wherein,
P₂ is the recovered pressure downstream of the cavity generator,
Pᵥ is the vapor pressure of liquid at the operating temperature,
Vₒ is average velocity of liquid at the cavity generator,
ρ is the density of liquid.

The cavitation number at which the inception of cavitation occurs is known as cavitation inception number Cᵥᵢ. Ideally, the cavitation inception occurs at Cᵥᵢ=1 and there are significant cavitational effects at Cᵥ value of less than 1. Further the dynamic behaviour of the cavities plays a significant role in intensification of physical and chemical processes.

Performance of a hydrodynamic cavitation reactor for a specific type of transformation depends on the cavitational conditions prevailing in the reactor. All the above mentioned studies have disclosed specific conditions for the application of hydrodynamic cavitation for a given process. However the above cited prior art does not teach how to design a hydrodynamic cavitation reactor for predetermined process intensification in diverse media.

Known in the prior art are devices and method for generation of hydrodynamic cavitation in a flowing fluid.

U.S. Pat. No. 5,492,654 discloses a hydrodynamic cavitation device for obtaining free dispersed systems, wherein the device comprises of a housing having an inlet opening, an outlet opening and internally accommodating a contractor, a flow channel provided with a baffle body and a diffuser installed in succession in said housing on the side of the inlet opening and connected with one another. The baffle body comprises at least two inter-connected elements to accomplish local contraction of flow in at least two sections in flow channel. Flow velocity is such maintained that the ratio of flow velocity at these sections to flow velocity at the outlet is at least 2.1 and degree of cavitation is at least 0.5. Degree of cavitation may be changed by changing the shape and distance between the baffles. However the free dispersed systems as per the patent are particularly limited to liquid-liquid & solid-liquid systems. It does not disclose the range of degree of cavitation that could be generated. It does not disclose which baffle shape or what baffle spacing gives what degree of cavitation. Hence it does not teach how to design or arrive at the hydrodynamic cavitation device/reactor for predetermined process intensification in diverse media.

U.S. Pat. No. 5,810,052 discloses a hydrodynamic cavitation device for obtaining a free disperse system comprising of a flow channel internally accommodating a single baffle body at or near the centre of flow channel or baffle body placed near the walls of channel. Degree of cavitation is claimed to be altered by different shapes of baffle body and by regulation of constriction ratio. The flow constriction ratio should be 0.8 and flow velocity at the contraction should atleast be 14 m/s. The free dispersed systems considered in the patent are particularly limited to liquid-liquid & solid-liquid systems. Although various shapes of the baffle are presented but no information is given which shape gives better or less degree of cavitation at any given geometric or operating conditions. Apart for maintaining flow velocity of at least 14 m/s, no information is given on range of operating pressure and temperature of the dispersed system and also the physico-chemical parameters of the liquids and solids under consideration. Hence it does not teach how to design or arrive at the hydrodynamic cavitation device/reactor for predetermined process intensification in diverse media.

U.S. Pat. Nos. 5,937,906, 6,012,492, 6,035,897 disclose method and apparatus for carrying out sono-chemical reactions using hydrodynamic cavitation on large scale. The device comprises of a flow through channel internally containing at least one element may either be a bluff body or a baffle which produces a local constriction of hydrodynamic flow thereby producing a cavitation cavern downstream of the element. The bluff body or the baffle of standard shapes like circular, elliptical, right-angle, polygonal and slots are presented. The device may be operated in recirculation mode. The patent discloses a hydrodynamic cavitation apparatus and a method of carrying out only those reactions which are previously classified as to sono-chemical reactions. The patent does not give any information about which shape of baffle body is better for sono-chemical reactions. The patent does not give any information about designing of hydrodynamic cavitational reactor for particular reactions (not necessarily Sono chemical but any reaction) for predetermined level of conversion. The teachings cannot be extended to or arrive at design of hydrodynamic cavitation reactor for carrying out predetermined physico-chemical transformation with predecided degree of conversion or process intensification.

U.S. Pat. Nos. 6,502,979, 7,086,777, 7,207,712 describes a device and method for creating hydrodynamic cavitation. The device comprises of a flow through chamber having an upstream portion and downstream portion wherein the downstream portion has cross-sectional area greater than the upstream portion and wherein the walls of the flow through chamber are removable and interchangeable mounted within the device. Baffle elements may have different shapes and sizes and are removable mounted within the flow through chamber for generation of cavitation downstream from the baffle element. The degree of cavitation is said to be changed by changing the shape, size and location of the baffle element. However it fails to explain the effect of these the parameters on the degree of cavitational in the reactor which is necessary and can be used for the useful transformations and can be used for design and optimization of the hydrodynamic cavitation reactor. The teachings cannot be extended to or arrive at design of hydrodynamic cavitation reactor for carrying out physico-chemical transformation to a predetermined level or intensify them.

Patent Application no WO 2007/054956 A1 describes an apparatus and method for disinfection of ship's ballast water, such as sea water, based on hydrodynamic cavitation. The cavitation chamber essentially being provided with single or multiple cavitation elements placed perpendicular to the direction of flow of fluid, said cavitation elements being spaced at uniform or non-uniform spacing and each said cavitation element having a fractional open area in the form of single or multiple orifices. However the method can not be used for the design of a cavitation reactors for transformations other than the treatment of ballast water as the effect of the type of the cavitation conditions has not been specifically related to the degree of disinfection.

All the above devices and method discussed in the prior art were used for a specific type of transformation with out due design considerations. None of them gives any information on the conditions of the cavitation/type of cavitation generated in the device. The reported prior arts also fail to teach a method for design of a hydrodynamic cavitation reactor with tailored cavitation conditions, which can be used to carry out a specific physico-chemical transformation. The type of cavitation conditions needed for specific physico-chemical transformation cannot be arrived at using the prior art and can not be extended seamlessly without undue experimentation by a person ordinarily skill in the art.

### OBJECTS OF THE INVENTION

The main object of the present invention is to provide a method for designing of hydrodynamic cavitation reactors to achieve tailored cavitating conditions in aqueous and non-aqueous media, for intensification of the physical and chemical processes.

Yet another object of the invention is to provide a method and a map of cavitation regimes generated using the said method for generating predetermined type of cavitation in a hydrodynamic cavitation reactor by a designer cavity (having specific size and behaving in a pre-decided dynamical manner) in the hydrodynamic cavitation reactors.

Yet another object of the invention is to provide a means of tailoring the cavity dynamics (i.e. generation, growth, oscillation and/or collapse of the cavity) in the hydrodynamic cavitation reactor by altering the constructional features of a reactor and the operating conditions.

Yet another object of the invention is to provide a method for controlling behavior of a cavity by altering the turbulence characteristics downstream of the point of cavity generation.

Yet another object of the invention is to provide a means of controlling the downstream turbulence to achieve a predetermined cavitation by synergistically combining the geometry of the flow modulator in the flow path of the reactants and the containment downstream of the said flow modulator and the nature of the reactants.

Yet another object of the invention is to provide hydrodynamic cavitation reactors with designer cavities for process intensification on industrial scale.

### DESCRIPTION OF FIGURES

FIG. 1 shows Cavitation regime map for various design of cavitation chamber. It plots velocity through the cavity generator against the % of cavitation and cavitation number.

FIG. 2 shows the cavitation regime map for non-aqueous systems. It shows effect of changing liquid density on extent and type of cavitation.

FIG. 3 shows the variation in active cavitation and stable cavitation as a function of density and viscosity.

FIG. 4 shows numerically evaluated cavitational conditions for examples included in the patent.

### DETAILED DESCRIPTION OF THE INVENTION

The present invention relates to designing of hydrodynamic cavitation reactors to achieve tailored cavitating conditions in aqueous and non-aqueous media, for intensification of the physical and chemical processes. In the present invention, a novel and useful and operational relationship is established between the effects of constructional features of the hydrodynamic cavitation reactors and operating conditions on the cavitation conditions (cavity dynamics and intensity of cavitation) followed by the use of such relationship to design hydrodynamic cavitation reactors to arrive at predetermined cavitation conditions for intensification of the physical and chemical processes.

A hydrodynamic cavitation reactor comprises of a cavity generator, cavity diverter and turbulence manipulator wherein the cavity generator/cavity diverter is a flow modulator of various shapes and sizes. The turbulence manipulator comprises of variety of geometric elements capable of changing the scale and intensity of turbulence making the cavity to grow, oscillate and/or collapse resulting into oscillatory, transient or multi-collapse cavity behavior most suited for a desired physico-chemical transformation. The flow modulator can be an orifice and/or orifices (sharp or profiled) with circular or rectangular or triangular or any other suitable shape or a venturi having converging and diverging section with suitable converging or diverging angles.

Thus in accordance of this invention, to start with, CFD simulation of various constructional features of the configuration of a flow modulator and range of operating conditions are performed using any commercial CFD code, like FLUENT 6.2 with RNG k-ε turbulence model. The flow information like static pressure, turbulent kinetic energy and frequency obtained from CFD simulations is used on cavity dynamics simulations. Cavity dynamics simulations are based on bubble dynamics models like Rayleigh-Plesset equation and Tomita-Shima equation.

Cavities which produce an instantaneous pressure of atleast 10 times higher than the maximum pressure in the system can produce cavitational effect and are termed as active cavities and fraction of active cavities is estimated as:

$$Active cavities ( \% ) = \frac{\begin{matrix}Number of cavities producing \\ cavitational effect\end{matrix}}{\begin{matrix}Total number of cavities \\ injected in the domain\end{matrix}} \qquad ( 2 )$$

The Cavitation conditions generated are represented as % cavitational activity, defined as cavities showing stable or transient collapse behavior and not simple dissolution characteristics. The % Transient cavitation indicates out of the total cavitational activity what % of cavities show transient behavior (undergoes collapse in single volumetric expansion and contraction cycle) and similarly the % stable cavitation (undergoes collapse in single volumetric expansion and contraction cycle) indicates out of the total cavitational activity what % of cavities show oscillatory behavior.

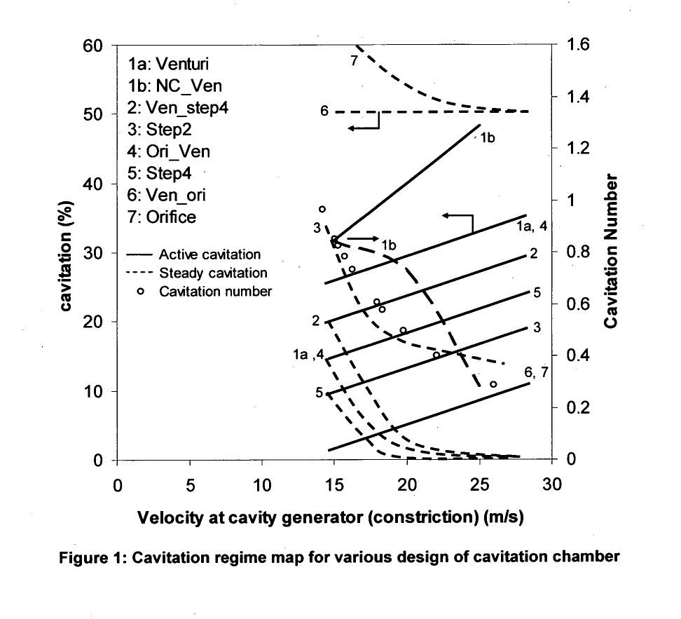

The effect of the variation in the configuration of the flow modulator and the operating conditions (Table 1) on the cavitation conditions in the hydrodynamic cavitation reactors is mapped on the basis of a defined parameter such as the Cavitation Number (FIG. 1) defined for water like fluids. The velocity of the flow at the flow modulator in the (FIG. 1) map represents the effect of the various constructional features of the flow modulator and the range of operating conditions considered. In one aspect of the invention, relationships are established and validated between the intensity and type of cavitation occurring in the cavitational device with a range of geometries and operating conditions as illustrated in Table 1 and FIG. 1. In a related aspect of the invention, a regime map similar to FIG. 1 will be utilized to identify the desired type of cavitation required for specific targeted process intensification and then reactors are designed to achieve the desired and predetermined process intensification.

FIG. 1 establishes that for a particular (identical Cavitation number, arrived at with different geometrical configurations and operating conditions) cavitation number (degree of cavitation) there is a quantifiable difference in the cavitation conditions (transient or stable or active) inside the hydrodynamic cavitation reactor which can be used to design hydrodynamic cavitation reactors to achieve tailored cavitating conditions in aqueous and non-aqueous media, for intensification of diverse physical and chemical processes

FIG. 1 can be utilized to arrive at the effect of the constructional features of the hydrodynamic cavitation reactor and the operating conditions represented by the velocity of the flow as a result of the presence of flow modulator. As will be shown from in the accompanying examples a clear relationship has been established using this method proposed in the invention between the type of transformation and the cavitation conditions prevailing in the reactor depending on the geometry and the operating conditions, which can facilitate and/or intensify the said physics/chemistry behind the transformation. Thus FIG. 1 can be used to design cavitation reactors for predetermined ranges of operating conditions to get the desired cavitation conditions/type of cavitation for a specific desired type of transformation.

For example, the effect of the flow velocity through the cavity generator on the cavitation conditions prevailing in the cavitation reactor. It can be seen from the FIG. 1 that the generation of cavitation (active cavitation) only starts after a threshold cavitation number of 1.0. With further decrease in cavitation number the cavitational event increase till cavitation number of 0.22. Any further decrease in cavitation number does not result in the increase in the cavitational events. This has been found for mostly for aqueous systems having predominantly water as the main fluid component.

It can also be seen from FIG. 1 that with an increase in the liquid velocity at the cavity generator the transient type of cavitation becomes more and more dominant, thereby decreasing the dominancy of the stable type of cavitation in the overall cavitation conditions prevailing. However, for cavitation number of 0.22 or below both transient and stable cavitation shows equal dependence in the overall cavitation conditions (% of active cavities).

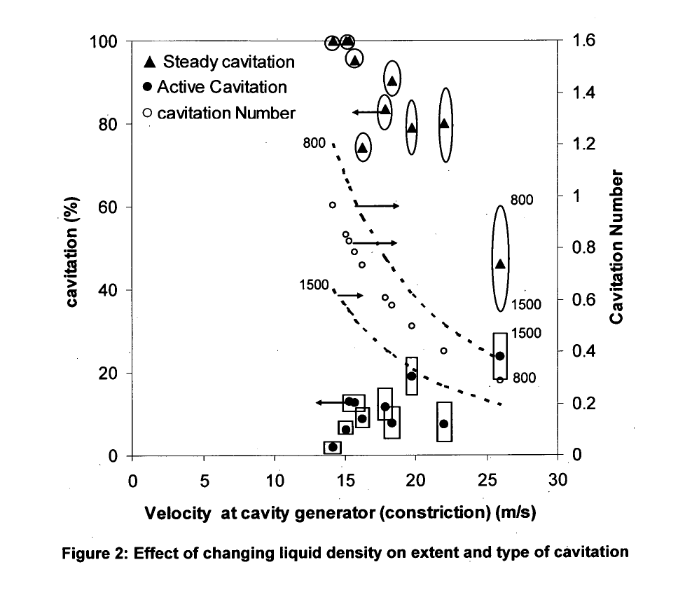

Cavitation map for non-aqueous systems is shown in FIG. 2. Non-aqueous system with reference to cavitating medium is essentially characterized by density, surface tension and viscosity significantly different than that for water. Present invention describes designing of cavitation system for any liquid or mixture of liquids having physico-chemical properties in range given below:

Density: 800 to 1500 kg/m³ (water: 1000 kg/m³)
Viscosity: 1 to 100 cP (water: 1 cP)
Surface tension: 0.01 to 0.075 N/m (water: 0.072 N/m)
Vapour pressure: 300 to 101325 N/m² at 30° C. (water: 4200 Pa)

The medium for the reactions/transformation can be selected from any suitable solvents having solubility/dispersing ability for the reactants and having physico-chemical properties in the same range as the reactants.

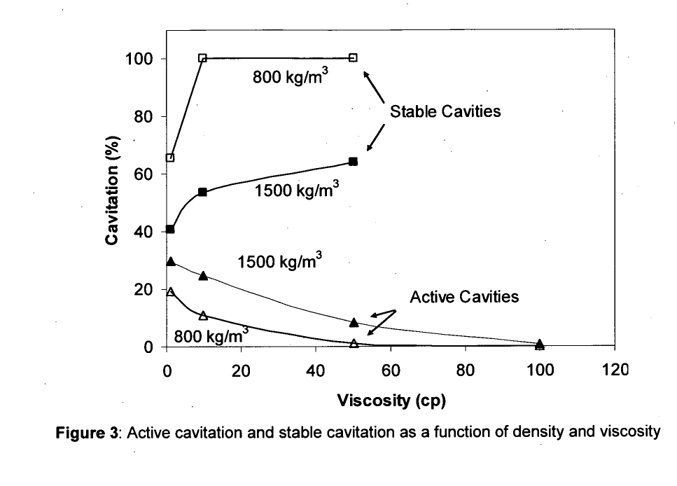

With increase in liquid density (in the said range from 800 kg/m³ to 1500 kg/m³) the extent of stable cavitation is seen to decrease (and transient cavitation increases) by almost 20%. Active cavities decrease as the liquid viscosity is increased and it ceases to exist beyond 100 cP (FIG. 3). Surface tension in the range of 0.01 to 0.075 N/m was seen to play not much a significant role in altering the extent or nature of cavitation for the two extreme cases of cavitation number of 1 and 0.37. The dimensionless parameter ‘Cavitation number’ takes into account the vapor pressure of the liquid thus variation in vapor pressure is directly reflected in cavitation map.

Thus hydrodynamic cavitation reactors may be designed to achieve cavitating conditions in aqueous and non-aqueous media for intensification of the physical and chemical processes, wherein the cavitation number is selected from the range
0.5 to 1.0 for stable cavitation for ‘Ven_ori’ and ‘Orifice’,
0.22 to 0.5 for transient cavitation for ‘Venturi’, ‘NC_ven’, ‘Ven_step4’, ‘Stepped2’, ‘Ori_Ven’, ‘Stepped4’,

0.22 to 0.5 for simultaneous stable and transient of cavitation for ‘Ven_ori’ and ‘Orifice’.

Thus in accordance with this invention a method of tailoring hydrodynamic cavitation reactors to achieve cavitating conditions in aqueous and non-aqueous media for intensification of the physical and chemical processes comprising steps of:
Selecting from stable and/or transient cavitation necessary for the targeted physical and/or chemical transformation respectively wherein,
the transient cavitation is selected for chemical transformation in homogenous system,
stable cavitation is selected for chemical transformation in heterogeneous and physical transformations in homogenous system, stable and transient both are selected for physical transformation in heterogeneous system;
Selecting the cavitation number from a range for the chosen physical or chemical transformation in the first step;
Selecting geometry of cavitation chamber from a regime map to maximize the active cavitation for the selected type of cavitation for the selected Cavitation number;
Determining the area of cavity generator within the said selected geometry and the said cavitation number for the volumetric flow rate to be processed using equation 3:

$$Area = \frac{Flow rate}{\sqrt{\frac{{P}_{2} - {P}_{V}}{\frac{1}{2} ρ \cdot {C}_{V}}}} \qquad ( 3 )$$

wherein, Area is area of cavity generator (m²), Flowrate is volumetric flowrate (m³/s), P₂ is pressure downstream to the cavity generator (Pa), Pᵥ is the Vapor pressure of the liquid to be processed for the selected transformation at the operating temperature (Pa), ρ is the density of liquid (kg/m³) and Cᵥ is the selected cavitation number; wherein optionally
   when the selected type of geometry of cavitation chamber is an orifice, optimization to maximize active cavitation is done by selecting multiple hole of smallest size such that a, which is ratio of perimeter of holes to flow area of holes, is maximized and sum of the flow area of multiple holes equals the said Area, such that the smallest size of hole is at least 50 times larger than the largest rigid/semi rigid particles in the heterogeneous phase, wherein the smallest size of hole is limited to 1 mm;
   if in a Liquid-Liquid heterogeneous system involving emulsification step that has a preceding chemical transformation, an additional criterion of Weber number=4.7 is chosen; wherein, Weber number (We) is defined as the ratio of inertial forces responsible for breakup to interfacial forces resisting the breakup;

$$We = \frac{{d}_{E} {v}^{{′}^{2}} ρ}{σ}$$

wherein, d_{E} is the size of emulsion, v′ is the turbulent fluctuating velocity, ρ is the density of liquid and σ is interfacial surface tension;
if the selected type of said geometry of cavitation chamber is a multiple orifice cavity generator, the spacing of the holes is obtained from:

dₛ=dₕ+4×10⁻⁴V_{J}
wherein, dₛ is the spacing between the holes (m); dₕ is minimum dimension of the hole (m) and V_{J} is the velocity of the liquid at cavity generator (m/s).

A regime map correlating maximum velocity of fluid or slurry through the cavitation chamber, cavitation number and percentage of active, transient and stable cavitation as in FIGS. 1, 2 & 4 is obtained by a process comprising steps:
establishing, over the geometry of cavitation chamber consisting of cavity generator, flow and turbulence modulator, the material continuity and the balance of momentum, turbulent kinetic energy and turbulent energy dissipation rate using appropriate equation consisting of fundamental variables like (P) pressure over the liquid, (u) velocity component in x direction, (v) velocity component in y direction, (w) velocity component in z direction, as per the frame of reference shown in table 1, (k) turbulent kinetic energy, (c turbulent energy dissipation rate, (ρ) liquid density, (a) liquid phase surface and interfacial tension, (μ) liquid viscosity;
wherein, continuity equation is:

$$\frac{\partial ρ}{\partial t} + ∇ \cdot ( ρ \overline{u} ) = 0 \qquad ( 4 )$$

wherein, momentum balance equation is:

$$\frac{\partial }{\partial t} ( ρ \overline{u} ) + ∇ \cdot ( ρ \overline{u} \overline{u} ) = - ∇ P - ∇ \cdot ( ρ {\overline{u}}^{′} {\overline{u}}^{′} ) + μ {∇}^{2} {\overline{u}}_{i} + ρ \overline{g} \qquad ( 5 )$$

wherein, turbulent kinetic energy equation is:

$$\frac{\partial }{\partial t} ( ρ k ) + \frac{\partial }{\partial {x}_{i}} ( ρ k {u}_{i} ) = \frac{\partial }{\partial {x}_{j}} [ ( μ + 0.09 ρ \frac{{k}^{2}}{ɛ} ) \frac{\partial k}{\partial {x}_{j}} ] - ( ρ {\overline{u}}_{i} {\overline{u}}_{j} \frac{\partial {u}_{j}}{\partial {x}_{i}} ) - ρɛ \qquad ( 6 )$$

wherein, turbulent energy dissipation rate equation is:

$$\frac{\partial }{\partial t} ( ρ ɛ ) + \frac{\partial }{\partial {x}_{i}} ( ρɛ {u}_{i} ) = \frac{\partial }{\partial {x}_{j}} [ ( μ + 0.069 ρ \frac{{k}^{2}}{ɛ} ) \frac{\partial ɛ}{\partial {x}_{j}} ] + 1.44 \frac{ɛ}{k} ( ρ {\overline{u}}_{i} {\overline{u}}_{j} \frac{\partial {u}_{j}}{\partial {x}_{i}} ) - 1.92 ρ \frac{{ɛ}^{2}}{k} \qquad ( 7 )$$

wherein, g̅ is the gravitational acceleration vector and the above equations are solved numerically to obtain P, k & ε;
Obtaining the ‘n’ number of likely paths taken by the cavities through the cavitation chamber;
wherein, n is significantly greater than 100;
wherein paths taken by cavity is obtained from Lagrangian equation:

$$\frac{\partial {u}_{P}}{\partial t} = {F}_{D} ( u - {u}_{P} ) + \frac{{g}_{x} ( {ρ}_{P} - ρ )}{{ρ}_{P}} \qquad ( 8 )$$

wherein, U_{P} is the cavity velocity, F_{D}(u-u_{P}) is drag force per unit mass of cavity, ρ_{P} is the density of cavity, gₓ is gravitational force in x direction (Table 1);
wherein, Lagrangian equation is solved numerically to obtain the time dependent co-ordinates of the cavity;
wherein, P_{Bulk}, k and ε □is obtained from the solution of balances at these co-ordinates obtained from Lagrangian equation (8);
obtaining the value of pressure amplitude (Pₐₘₚ), pressure frequency (f) and Instantaneous Pressure sensed by the cavity (P_{∞}) from relations:

$${P}_{amp} = 1 / 3 ρ k ; f = \frac{ɛ}{k} ; {P}_{∞} ( t ) = {P}_{Bulk} - {P}_{amp} sin ( 2 π ft ) ;$$

obtaining the cavity dynamics (cavity radius as a function of time) from cavity dynamics models using the above data of P_{∞}, Pₐₘₚ, f;
wherein, the cavity dynamics models are generally known as Rayleigh-Plesset family of equations e.g.

$$R ( \frac{{d}^{2} R}{d {t}^{2}} ) + \frac{3}{2} {( \frac{d R}{d t} )}^{2} = \frac{1}{{ρ}_{l}} [ {P}_{B} - \frac{4 μ}{R} ( \frac{d R}{d t} ) - \frac{2 σ}{R} - {P}_{∞} ] \qquad ( 9 )$$

wherein, t is time , R is radius of cavity at any instant, σ is liquid surface tension, μ is liquid viscosity, P_{B} is pressure inside the bubble;
Categorizing the cavities as active, stable and transient cavitation using the following criteria;
wherein, a cavity is active if pressure inside the cavity is more than 10 times the pressure at the inlet of cavitation chamber,
wherein, an active cavity is a stable cavity if final pressure is not equal to maximum pressure inside the cavity during its lifetime,
wherein, an active cavity is a transient cavity if final pressure is equal to the maximum pressure inside the cavity;
Calculating, for a given velocity, cavitation number, selected geometry (shape and size) of the cavitation chamber;
The percentage of active cavitation as number of active cavities/total number of cavities×100;
The percentage of stable cavitation as number of stable cavities/total number of active cavities×100;
The percentage of transient cavitation as number of transient cavities/total number of active cavities×100.

The above method has been used to tailor diverse geometries of cavitation chambers as:

i) A ‘Venturi’ comprising:
a cavity generator which is a portion or whole of minimum cross-sectional area in the cavitation chamber of circular or non circular shape which maximizes the value of a;
a flow modulator, which is a smooth converging section with an overall average angle of 52-56° upstream of the minimum cross sectional area names as the cavity generator and a smooth diverging section with an overall average angle of 20-25° downstream of cavity generator;

the said ‘Venturi’ consists of three co-axial sections placed sequentially in the direction of flow.

Convergence section is such that
The axis is a straight line
The cross-sectional area is circular throughout its length
The diameter of conduit decrease at the rate of 0.93 to 1.06 m per m in direction of flow

It terminates when the cross-sectional area equals the cross-sectional area of Throat section.

Throat section is such that
The axis is a straight line
The cross-sectional area of conduit is circular
The cross-sectional area is constant and is obtained from equation (3)
The length of section is equal to half of its diameter.

Divergence section is such that
The axis of the conduit is a straight line
The cross-sectional area of conduit is circular throughout its length
The diameter of conduit increase at the rate of 0.35 to 0.44 m per m in direction of flow
Its length is equal to 2.64 times the length of Convergence section.

ii) A ‘Ven_step4’ comprising:
a cavity generator which is a portion or whole of minimum cross-sectional area in the cavitation chamber of circular or non circular shape which maximizes the value of a;
turbulence modulator that is downstream of the said cavity generator having multiple sections of length(width) equal to maximum dimension of the cavity generator arranged along the longer axis parallel to the flow and held together forming a conduit;
flow modulator that is a smooth converging section with an overall average angle of 52-56° upstream of the cavity generator;

the said ‘Ven_step4’ consists of three co-axial sections placed sequentially in the direction of flow.

Convergence section is such that
The axis is a straight line
The cross-sectional area is circular throughout its length
The diameter of conduit decrease at the rate of 0.93 to 1.06 m per m in direction of flow
It terminates when the cross-sectional area equals the cross-sectional area of Throat section.

Throat section is such that
The axis is a straight line
The cross-sectional area of conduit is circular
The cross-sectional area is constant and is obtained from equation (3)
The length of section is half of its diameter.

Divergence section comprises of Multiple orifices such that
Each subsequent orifice plate is touching the previous orifice plate
Each Orifice plate has only one hole
All the holes in orifice plates are circular and co-axial with the axis of Throat section
Thickness of the each orifice plate is twice the length of Throat section
The diameter of subsequent orifice plate increases 0.35-0.44 times the thickness of each orifice plate
Length of this section is equal to 2.64 times the length of Convergence section.

iii) A Stepped2′ comprising:
a cavity generator which is a portion or whole of minimum cross-sectional area in the cavitation chamber of circular or non circular shape which maximizes the value of α;
turbulence modulator downstream and upstream of the said cavity generator which has sections of length (width) equal to half of the maximum dimension of cavity generator arranged along the longer axis parallel to the flow with increasing flow area and held together forming a conduit of increasing flow area having again an overall average angle of 52-56° upstream and 20 -25° downstream;

the said ‘Stepped2’ consists of three co-axial sections placed sequentially in the direction of flow.

Convergence section comprises of Multiple orifices such that
Each subsequent orifice plate is touching the previous orifice plate
Each Orifice plate has only one hole
All the holes in orifice plates are circular and co-axial with the axis of Throat section
Thickness of the each orifice plate is equal to the length of Throat section
The diameter of hole in the subsequent orifice plate decrease 0.93-1.06 times the thickness of each orifice plate
It terminates when the area of hole equals the cross-sectional area of Throat section.

Throat section is such that
The axis is a straight line
The cross-sectional area of conduit is circular
The cross-sectional area is constant and is obtained from equation (3)
The length of Throat section is half of its diameter.

Divergence section comprises of Multiple orifices such that
Each subsequent orifice plate is touching the previous orifice plate
Each Orifice plate has only one hole
All the holes in orifice plates are circular and co-axial with the axis of Throat section
Thickness of the each orifice plate is equal to the length of Throat section
D The diameter of subsequent orifice plate increases 0.35-0.44 times the thickness of each orifice plate
Length of this section is equal to 2.64 times the length of Convergence section

iv) A ‘Ori_Ven’, comprising:
a cavity generator which is a portion or hole of minimum cross-sectional area in the cavitation chamber of circular or non circular shape which maximizes the value of a;
flow modulator which is a smooth diverging section with an overall average angle of 20-25° downstream of the cavity generator;

the said ‘Ori_Ven’ consists of two co-axial sections placed sequentially in the direction of flow.

Throat section is such that
The axis is a straight line
The cross-sectional area of conduit is circular
The cross-sectional area is constant and is obtained from equation (3)
The length of section is equal to half of its diameter

Divergence section is such that
The axis of the conduit is a straight line
The cross-sectional area of conduit is circular throughout its length
The diameter of conduit increase at the rate of 0.35 to 0.44 m per m in direction of flow
Its length is equal liquid flowrate (m³/s)/area of throat section (m²) *0.001 m.

v) A ‘Stepped4’ comprising:
a cavity generator which is a portion or whole of minimum cross-sectional area in the cavitation chamber of circular or non circular shape which maximizes the value of α;
turbulence modulator downstream and upstream of the said cavity generator as an assembly of multiple sections of length (width) equal to the maximum dimension of the said cavity generator arranged in a decreasing and increasing order respectively in terms of the flow area having an overall average angle of 20-25° and 52-56° respectively; the said ‘Stepped4’ consists of three co-axial sections placed sequentially in the direction of flow.

Convergence section comprises of Multiple orifices such that
Each subsequent orifice plate is touching the previous orifice plate
Each Orifice plate has only one hole
All the holes in orifice plates are circular and co-axial with the axis of Throat section
Thickness of the each orifice plate is twice the length of Throat section
The diameter of hole in the subsequent orifice plate decrease 0.93-1.06 times the thickness of each orifice plate
It terminates when the area of hole in the orifice plate equals the cross-sectional area of Throat section.

Throat section is such that
The axis is a straight line
The cross-sectional area of conduit is circular
The cross-sectional area is constant and is obtained from equation (3)
The length of Throat section is half of its diameter

Divergence section comprises of Multiple orifices such that
Each subsequent orifice plate is touching the previous orifice plate
Each Orifice plate has only one hole
All the holes in orifice plates are circular and co-axial with the axis of Throat section
Thickness of the each orifice plate is twice the length of Throat section
The diameter of subsequent orifice plate increases 0.35-0.44 times the thickness of each orifice plate
Length of this section is equal to 2.64 times the length of Convergence section.

vi) A Ven_Orr comprising:
a cavity generator which is portion of minimum cross-sectional area in the cavitation chamber of any shape which maximizes the value of a;
flow modulator which is a smooth converging section with an angle of 52-56° to the upstream of cavity generator;

the said ‘Ven_Ori’ consists of two co-axial sections placed sequentially in the direction of flow.

Convergence section is such that
The axis is a straight line
The cross-sectional area is circular throughout its length
The diameter of conduit decrease at the rate of 0.93 to 1.06 m per m in direction of flow
It terminates when the cross-sectional area equals the cross-sectional area of Throat section.

Throat section is such that
The axis is a straight line
The cross-sectional area of conduit is circular
The cross-sectional area is constant and is obtained from equation (3)
The length of section is equal to half of its diameter.

vii) An ‘Orifice’ comprising:
a cavity generator which is a portion or whole of minimum cross-sectional area in the cavitation chamber of circular or non circular shape which maximizes the value of α.

The said Orifice consists of Throat section such that
The axis is a straight line
The cross-sectional area of conduit is circular
The cross-sectional area is constant and is obtained from equation (3)
The length of section is equal to half of its diameter.

viii) A ‘NC_Ven’ comprising:
a cavity generator which is a portion or whole of minimum cross-sectional area in the cavitation chamber of non circular shape which maximizes the value of α
flow modulator, which is a smooth converging section with an overall average angle of 52-56° upstream of cavity generator and a smooth diverging section with an average overall angle of 20-25° downstream of cavity generator; maintaining the same or different yet a non-circular shape downstream of the said cavity generator.

The invention is now illustrated with non-limiting examples of the design of reactors for the use of hydrodynamic cavitation involving process intensification in specific physical, chemical or biological transformations like Water Disinfection by Disruption of bacteria, Degradation of Rhodamine, Toluene Oxidation, Biofouling in Cooling Towers, Esterification of Fatty Acids and Release of Soluble Carbon. Examples related to the effect of geometry, energy consumption, cavitation optimization have also been included.

### EXAMPLES

The design features, operating conditions, cavitation conditions and the effect of these cavitation conditions on various transformations are listed in Table 2a. These cavitating devices (orifice plates of different configurations which were first simulated as shown in Table 2a) have been fabricated and tested with experiments to validate FIG. 1 for the design of the hydrodynamic cavitation reactor.

FIG. 1 has been validated and then used to design reactors to carry out specific process intensifications and illustrate the application of FIG. 1 as described above.

### Example 1

### Geometrical Analysis of Cavitational Chamber

Various geometries of cavitation chambers were designed to handle the representative liquid flowrate of 2.5×10⁻⁴ m³/s and cavitation number of 0.5. Above parameters are selected for illustration purpose only but the presented methodology and the designs obtained therein can be utilized for range of these operating and design parameters. Area of the cavity generator was calculated from equation (3) for above mentioned representative liquid flowrate (2.5×10⁻⁴ m³/s) and selected cavitation number (0.5) as 1.26×10⁻⁵ m². Based on the present methodology several shapes of cavitational device were obtained & were analyzed for cavitational behavior.

The pressure drop predicted for various designs are given in Table 3. It is seen that for a given liquid flowrate, the lowest pressure drop (0.475 atm) occurs in venturi while highest pressure drop (3.15 atm) occurs in orifice. The pressure drop in case of ‘ori_ven’ is lower (1.55 atm) than the pressure drop in ‘ven_ori’ (2.13 atm).

Table 3 shows % of active cavities of total cavities injected for various designs. It is seen that % of Active cavities is higher when downstream section is divergent (venturi/stepped) instead of sudden expansion as that in orifice.

Table 3 details the extent of active and transient cavities produced in several designs. Table 3 presents percentage of active cavities per unit pressure drop and percent of transient cavities per unit pressure drop obtained from current invention. Using present methodology it is possible to quantify the cavitational behavior of cavitational device and an optimized geometry and operating parameter can be arrived at for a given physico-chemical transformation.

Cavitation regime map for various designs is generated based on the presented methodology and is shown in FIG. 1. Solid lines indicate the extent of active cavities while the dotted lines indicate the extent of stable cavities. Using the cavitation regime map operating parameters (cavitation number) can be decided for any design of cavitation element. Although FIG. 1 shows cavitation regime map for water like substance but it can be altered for liquid substantially different in density, viscosity, surface tension and vapor pressure based on the discussion made earlier here (FIG. 2).

### Example 2

### Potable Water Disinfection/Disruption of Bacteria Using Hydrodynamic Cavitation

Microbial cell disruption is carried out for several applications like water disinfection, waste water treatment, avoiding bio-fouling, enzyme recovery etc. Microbial cell gets disrupted when cavities collapse (transient cavitation) or undergo rapid volumetric oscillations (stable cavitation) near the microbial cell. If the imposed stress, produced either by transient or stable cavitation, is significantly greater than the cell strength cell wall gets disrupted. Thus both the types of cavitation are likely to assist the extent of cell disruption. Microbial disinfection occurs due to physical effects of cavitation in a heterogonous system. Thus, both the stable and transient cavitation should be maximized for microbial cell disruption. From regime map shown in FIG. 1, a cavitation number is selected in the range of 0.22 to 0.5 which gives highest stable cavitation for orifice. For a cavitation number of 0.28 selected from the above range, for a flowrate of 6.73×10⁻⁴ m³/s the area of holes in orifice was calculated from equation (3) as 2.55×10⁻⁵ m². This area of hole corresponds to a single hole of diameter 5.70 mm. Since the selected cavitation chamber was an orifice plate, we need to maximize the value of a (ratio of perimeter of holes to open area). We select a limiting value of 1 mm which gives highest value of α. Accordingly orifice plates were designed and fabricated with 33 holes of 1 mm diameter. The performance characteristics of the cavitation element (orifice plate) at different inlet pressure are shown in Table 2b. It can be seen from Table 2a that the intensity of cavitation (% of active cavities) increases with increase in the inlet pressure due to which the percentage of disinfection also increases. A four fold increase in the inlet pressure (from 1.72 bar to 5.77 bar) has resulted in 13 fold increase in the active cavitation thereby resulting in 50% increase in the disinfection. As said earlier the type of cavitation (transient or stable) has a significant effect on the disinfection of water. Water disinfection study carried out at low liquid velocities ˜14 m/s (w-1) showed that although very few transient cavities are present resulting in substantial disinfection of about 60% from oscillatory (stable) cavities. Further, as the quantum of transient cavitation increased by 53% the disinfection was seen to increase by 50% indicating a near one to one correspondence between the transient cavitational effect and disinfection. Thus by designing & operating cavitational device in stable cavitation or transient cavitation based on FIGS. 1 & 4, required effects are achieved in terms of physical transformation. Thus a tailored cavitation reactor for microbial cell disruption in heterogeneous system has been designed to operate in stable and transient cavitation wherein the cavitation number is selected from 0.22 to 0.5 preferably 0.28 for a flowrate of 6.73×10⁻⁴ m³/s , wherein the area of holes in orifice is 2.55×10⁻⁵ m² corresponding to a single hole of diameter 5.70 mm, wherein the smallest hole diameter is chosen to maximize the value of a but to a limiting value when hole diameter is 1 mm, thereby amounting to 33 holes to achieve the required total flow area, and active cavitation of 39%, out of which the extent of stable cavitation is 46% resulting in 86% disruption of cells takes place.

### Example 3

### Degradation of Rhodamine Using Hydrodynamic Cavitation

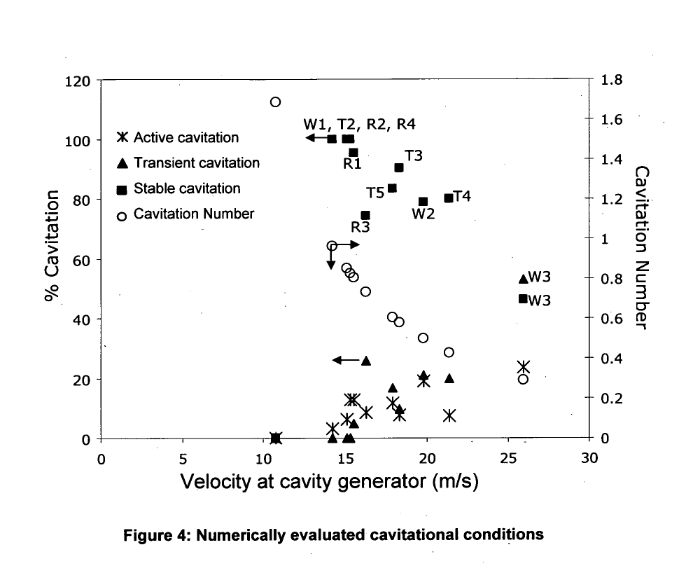

Rhodamine is an aromatic amine dye, commonly used in textile industries. It becomes necessary to decolorize the waste stream which contains such pollutants. Cavitation breaks the chromophore of such molecules thus decolorizes the waste effluent stream. This is physical transformation in homogenous system. Hence stable cavitation should be maximized for such a transformation. From regime map shown in FIG. 1, the cavitation number is should be in the range of 0.5 to 1.0 which gives highest stable cavitation for orifice. A cavitation number of 0.78 is selected from the chosen range of cavitation number and open area of orifice is calculated to be 2.59×10⁻⁵ m² from equation (3) for flowrate of 4.08×10⁻⁴ m³/s. This open area corresponds to a single hole of diameter 5.7 mm. Since the selected cavitation chamber was an orifice plate, we need to maximize the value of a (ratio of perimeter of holes to open area). We select a limiting value of 1 mm which gives highest value of α. Along with this geometry few other design of orifice plate with varying value of α (2 & 1.33) were also designed and fabricated to compare the ability (for details see Table 2a) to generate hydrodynamic cavitation. The performance characteristics of the three different orifice plates for same inlet pressure are show in Table 2a. It can be seen from Table 2b that for the same inlet pressure the percentage degradation of Rhodamine varies with the geometry of the cavitation element. The percentage degradation increased with an increase in the value of α (Table 2a). Comparison of R-1 and R-2 (FIG. 4) indicate that, with same amount of active cavities, the occurrence of 5% transient cavitation can increase the degradation by approximately 50%. Similarly, the comparison of R-3 and R-2 configuration reveals that, though there is a 32% decrease in the quantum of active cavitation for R-3 configuration (FIG. 4), the decrease in the degradation is marginal (1%). This can be attributed to the increase in the quantum of transient cavitation, in case of R-3, by approximately 25%. This clearly indicates the type of cavitation generated & predicted by FIG. 4 and obtained by the constructional features of the cavitational elements plays an important role in the Rhodamine degradation, which is based on the breakage of molecular bonds, resulting into the breakage of chromophore and resulting discoloration. It is seen that orifice plate designed on the basis of given methodology, with maximum value of a, gave highest extent of transformation as compared to the other design for the reasons mentioned above. Thus a tailored cavitation reactor for Rhodamine degradation has been designed to operate in stable cavitation wherein the cavitation number is selected from from 0.5 to 1.0 preferably 0.78 to achieve the highest stable cavitation for flowrate of 4.08×10⁻⁴ m³/s wherein area of holes in orifice is 2.59×10⁻⁵ m², corresponding to a single hole of diameter 5.7 mm, wherein the smallest hole diameter is chosen to maximize the value of a but to a limiting value when hole diameter is 1 mm, thereby amounting to 33 holes to achieve the total flow area and stable cavitation of 95% resulting in 17% degradation of Rhodamine.

### Example 4

### Toluene Oxidation Using Hydrodynamic Cavitation

The oxidation of alkylarenes to the corresponding aryl carboxylic acids is an industrially important process. Industrially such oxidations are carried out using dilute HNO₃ or air under high temperature and high-pressure conditions. This is a heterogeneous system and requires high agitation speeds to achieve sufficient blending of reactants. Hydrodynamic cavitation produces fine emulsion of reactants and also provides radicals for oxidation of alkylarenes. Hydrodynamic cavitation was used to carry out oxidation of toluene. This is chemical transformation in heterogeneous system. Hence stable cavitation should be maximized for such a transformation. From regime map shown in FIG. 1, the cavitation number is should be in the range of 0.5 to 1.0 which gives highest stable cavitation for orifice. A cavitation number of 0.78 is selected from the chosen range of cavitation number and open area of orifice is calculated to be 11.3×10⁻⁵ m² from equation (3) for flowrate of 22.2×10⁻⁴ m³/s. This open area corresponds to a single hole of diameter 12 mm. Since the selected cavitation chamber was an orifice plate, we need to maximize the value of α (ratio of perimeter of holes to open area). To maximize the value of smallest holes are selected of at least 50 times the size of largest rigid/semi rigid particles in the heterogeneous phase, yet limited to a value of 1 mm. In accordance with the method described for a liquid-liquid heterogeneous system, the maximum size of dispersed phase is obtained by Weber number criterion (We=4.7). For an turbulent fluctuating velocity of 2.5 m/s the size of dispersed phase is obtained from Weber number as 0.051 mm. Thus the limiting value of holes should be (50×0.0051) 2.51 mm rounded to 3 mm for ease of fabrication. Thus an orifice with 16 holes with 3 mm diameter was & designed and fabricated. Along with this design one more design with value of a of 2 was fabricated to compare the performance. Table 2a shows the details of the geometry and operating conditions used. Comparison of the case T-2 and T-4 reveals that a 20% increase in the quantum of active (FIG. 4) cavitation results in 26% increase in the conversion. The role of stable cavities are correlated as this reaction requires physical (emulsification, controlled by oscillatory cavity) and chemical (oxidation, controlled by transient cavity) effects for the overall reaction progress & intensification. A tailored cavitation reactor for Toluene oxidation in a heterogeneous liquid-liquid system, has been designed to operate in maximized stable cavitation wherein the cavitation number is selected from 0.5 to 1.0 preferably cavitation number of 0.78, more preferably cavitation number of 0.5 for maximized percentage of active cavitation for flowrate of 22.2×10 ⁻⁴ m³/s wherein the area of holes in orifice is 11.3×10⁻⁵ m² which corresponds to a single hole of diameter 12 mm, wherein optionally the smallest diameter of hole is chosen to maximize the value of α but to a limiting value when diameter of hole is 1 mm or at least 50 times the size of largest rigid/semi rigid particles, resulting in a minimum diameter of hole to ˜2.51 mm thereby amounting to orifice plate with 3 mm diameter of 16 holes to achieve. Stable cavitation of 90.3% resulting in 53% oxidation of toluene or at cavitation number of 0.4 resulting in stable cavitation of 80% to achieve 54% oxidation of toluene.

### Example 5

### Eliminating Bio-Fouling in Cooling Tower Using Cavitation

Microbial growth (algae/fungi) in cooling tower water leads to bio-fouling in cooling towers and related heat exchange equipments. Stable and transient cavitation should be maximized for microbial cell disruptionand therefore the cavitation chamber should give the highest active cavitation for such an application. Hence in accordance with the method described, a cavitation number is selected in the range of 0.5 to 1.0 to give the highest active cavitation for venturi with least pressure drop. For a cavitation number of 0.8 selected from the above range, for a flowrate of 3.14×10⁻² m³/s the area of throat in venturi was calculated from equation (3) as 12.57×10 m². The cavitation number was kept at 0.8 by maintaining the discharge pressure at 2.5 atm and velocity equal to 25 m/s. The selected design of cavitation chamber for stated operating parameters produces 26% of active cavitation and 10% of transient cavitation. Table 4 shows the decrease in bacterial count from 1,00,000 CFU/ml to 0 CFU/ml in a period of 13 days from the water that is circulated in cooling loop.

Thus a tailored cavitation reactor for eliminating biofouling in heterogeneous system has been designed to operate in stable and transient cavitation wherein the cavitation number is selected from 0.5 to 1 preferably 0.8 for a flowrate of 3.14×10⁻² m³/s, wherein the area of cavity generator in venturi is 12.57×10 m² corresponding to a cavity generator of diameter 40 mm, and active cavitation of 26%, out of which the extent of transient cavitation is 10% resulting in 100% decrease in bacterial count.

### Example 6

### Esterification of C₈/C₁₀ Fatty Acids Using Hydrodynamic Cavitation

Hydrodynamic cavitation was used to carry out esterification of fatty acid with methanol to produce methyl esters. Stable cavitation for such a chemical transformation in a heterogeneous system needs to be maximized for such a transformation. Thus in accordance with the method the cavitation number should be in the range of 0.5 to 1.0 which gives highest stable cavitation for orifice. A cavitation number of 0.78 is selected from the chosen range of cavitation number and open area of orifice is calculated to be 11.3×10⁻⁵ m² from equation (3) for a flowrate of 22.2×10⁻⁴ m³/s. This open area corresponds to a single hole of diameter 12 mm. Since the selected cavitation chamber is an orifice plate, he value of α (ratio of perimeter of holes to open area) needs to be maximized for which the smallest holes are selected of at least 50 times the size of largest rigid/semi rigid particles in the heterogeneous phase, yet limited to a value of 1 mm. In accordance with the method described for a liquid-liquid heterogeneous system, the maximum size of dispersed phase is obtained by Weber number criterion (We=4.7). For an turbulent fluctuating velocity of 2.5 m/s the size of dispersed phase is obtained from Weber number as 0.051 mm. Thus the limiting value of holes should be (50×0.0051) 2.51 mm rounded to 3 mm for ease of fabrication. Thus, an orifice was tailored with 16 holes with 3 mm diameter. On operating the orifice design based on the method stated above 90% of the C₈/C₁₀ fatty acids are converted to methyl esters in 210 mins.

A tailored cavitation reactor for Esterification of C₈/C₁₀ fatty acids in a heterogeneous liquid-liquid system, has been designed to operate in maximized stable cavitation mode wherein the cavitation number is selected from 0.5 to 1.0 preferably cavitation number of 0.78, more preferably cavitation number of 0.5 for maximizing percentage of active cavitation for flowrate of 22.2×10 ⁻⁴ m³/s wherein the area of holes in orifice is 11.3×10⁻⁵ m² which corresponds to a single hole of diameter 12 mm, wherein optionally the smallest diameter of hole is chosen to maximize the value of α but to a limiting value when diameter of hole is 1 mm or at least 50 times the size of largest rigid/semi rigid particles, resulting in a minimum diameter of hole to ˜2.51 mm thereby amounting to orifice plate with 3 mm diameter of 16 holes to achieve. Stable cavitation of 90.3% resulting in 90% esterification of C₈/C₁₀ fatty acid in 210 mins at a cavitation number of 0.78.

### Example 7

### Release of Soluble Carbon for Activated Sludge Treatment Using Hydrodynamic Cavitation

Using hydrodynamic cavitation the soluble carbon is obtained for activated sludge treatment from the disruption of activated biomass in the system. For such an application transient cavitation needs to be maximized to achieve release of soluble carbon in an efficient manner. In accordance with the method, a cavitation number is selected in the range of 0.22 to 0.5 which gives highest transient cavitation for venturi with least pressure drop (table 3). For a cavitation number of 0.5 selected from the above range of cavitation number and a flowrate of 2.23×10 m³/s the area of holes in orifice was calculated from equation (3) as 1.18×10⁻⁵ m². This area of hole corresponds to a throat diameter of 3.88 mm (˜4 mm) of the ventury. On operating a tailored venturi based on the method 2000 ppm of soluble carbon is released within 10 mins of operation.

Thus, by designing & operating cavitational device in transient cavitation, based on FIG. 1 & 4, required effects are achieved in terms of physical transformation. Thus, a tailored cavitation reactor for the release of soluble carbon from biomass, disruption in heterogeneous system has been designed to operate in transient cavitation wherein the cavitation number is selected from 0.22 to 0.5 preferably 0.55 for venturi with a flowrate of 2.23×10 m³/s , wherein the area of cavity generator in venturi is 1.18×10⁻⁵ m² corresponding to a cavity generator of diameter 4 mm, and active cavitation of 30%, out of which the extent of transient cavitation is 96% resulting in release of 2000 ppm of soluble carbon from the disrupted biomass.

In conclusion, in the examples cited above both types of cavitation i.e. transient cavitation & stable cavitation are seen to bring about the physico-chemical transformation depending on the mechanism of transformation. Microbial disinfection (water disinfection) & Rhodamine degradation is brought about dominantly by stable cavitation, while transient cavitation is necessary especially when intense cavitation is required (Release of soluble of carbon) and when changes are required at the molecular level (Toluene oxidation). Cavitation can be tailored (designer cavity) to achieve specific transformations that require predetermined specific minimum energy of transformation and the geometry of a cavitation element and the operating conditions can be tailored to create a dominant specific type of cavitation i.e. size of the cavity, transient and/or stable behavior of the cavity and the number of cavitationally active events. The examples clearly demonstrate the power of the present invention that facilitates cavitational mapping for designing of cavitational reactors to achieve pre-determined physico-chemical transformations, for example:
For a cavitation number in the range of 0.5-1, stable type of cavitation is dominant mainly responsible for physical effects in fluids having water like properties,
For a cavitation number in the range of 0.5-0.22 transient type of cavitation is more dominant which is mainly responsible for chemical effects in water like fluids,
For a cavitation number below 0.22 both transient and stable type of cavitation shows equal dominance and is useful for transformations requiring both the physical and chemical effects in overall transformations in water like fluids.

TABLE 1
various design of cavitational chamber

| S. No. | Name | Design | Details |
|---|---|---|---|
| 1(a) 1(b) | Venturi & NC_ven | 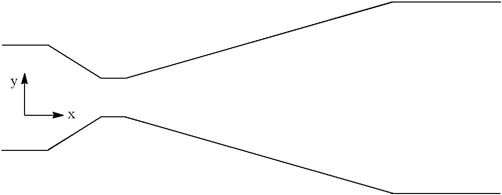 | Smooth convergence, smooth divergence circular & non-circular venturi |
| 2. | Ven_step4 | 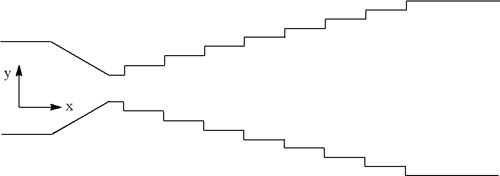 | Smooth convergence, stepped divergence, step length = 4 mm |
| 3. | Stepped2 | 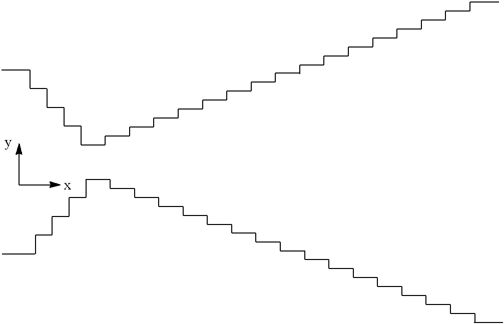 | Stepped convergence, stepped divergence Step length = 2 mm |
| 4. | Ori_Ven | 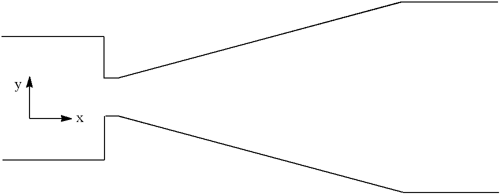 | Sudden convergence, Smooth divergence |
| 5. | Stepped4 | 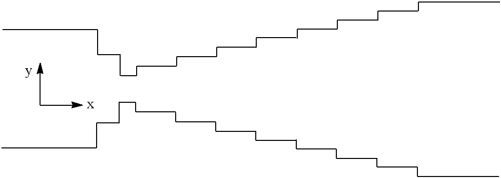 | Stepped convergence, stepped divergence Step length = 4 mm |
| 6. | Ven_Ori | 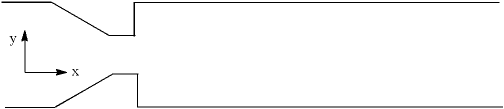 | Smooth convergence, Sudden divergence |
| 7. | Orifice | 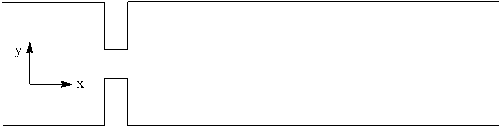 | Sudden convergence, Sudden divergence |

TABLE 2
(a):-Estimation of cavity dynamics for various geometries
(b): Extent of transformations

| (a) | | | | | | | | | | | | | |
|---|---|---|---|---|---|---|---|---|---|---|---|---|---|
| | Geometric Details of the cavitation | | | | | | | | | | | | |
| | element (orifice plate) | | | | | | | | | | | | |
| | | Size | | | | | | | Particle | | | | |
| | No. | of | | | | Operating Parameter | | | Tracking | | | | (b) |
| | of | hole | % | | | Inlet | Pipe | Orifice | Total | Cavity Dynamics | | | % |
| Sr. | Holes | (do), | Free | | | Pressure | Velocity | Velocity | Particle | Active | T | OS | Trans- |
| No. | (n) | mm | area | Alpha | Beta | P1, (bar) | V_{P} (m/s) | V_{P} (m/s) | tracked | (%) | (%) | (%) | Formation |

WATER DISINFECTION (POTABLE WATER)

| | | | | | | | | | | | | | |
|---|---|---|---|---|---|---|---|---|---|---|---|---|---|
| W-1 | 33 | 1 | 2.28 | 4 | 0.02 | 1.72 | 0.3 | 14.22 | 165 | 3 (1.82%) | — | 3 (100%) | 60 |
| W-2 | | | | | | 3.44 | 0.43 | 19.8 | 100 | 19 (19%) | 4 (21%) | 15 (78.95%) | 73 |
| W-3 | | | | | | 5.77 | 0.55 | 25.98 | 165 | 39 (23.6%) | 21 (53.84%) | 18 (46.15%) | 86 |
| W-4 | | | | | | 7 | | | | | | | — |

RHODAMINE DEGRADATION

| | | | | | | | | | | | | | |
|---|---|---|---|---|---|---|---|---|---|---|---|---|---|
| R-1 | 33 | 1 | 2.28 | 4 | 0.02 | 2.06 | 0.33 | 15.75 | 165 | 21 (12.73%) | 1 (4.76%) | 20 (95.24%) | 17 |
| R-2 | 8 | 2 | 2.22 | 2 | 0.02 | | 0.311 | 15.34 | 156 | 20 (12.82%) | — | 20 (100%) | 12 |
| R-3 | 16 | 3 | 9.97 | 1.33 | 0.1 | | 1.46 | 16.29 | 406 | 35 (8.62%) | 9 (25.71%) | 28 (74.29%) | 11 |
| R-4 | 8 | 5 | 13.85 | | | | 1.87 | 15.73 | 344 | | | | — |

TOLUENE OXIDATION

| | | | | | | | | | | | | | |
|---|---|---|---|---|---|---|---|---|---|---|---|---|---|
| T-1 | 16 | 3 | 9.97 | 1.33 | 0.1 | 0.98 | 0.94 | 10.75 | 406 | — | — | — | 42 |
| T-2 | | | | | | 1.96 | 1.32 | 15.11 | 406 | 25 (6.16%) | — | 25 (100%) | 43 |
| T-3 | | | | | | 2.94 | 1.6 | 18.34 | 406 | 31 (7.63%) | 3 (9.68%) | 28 (90.32%) | 53 |
| T-4 | | | | | | 3.92 | 1.8 | 22.05 | 406 | 30 (7.39%) | 6 (20%) | 24 (80%) | 54 |
| T-5 | 8 | 2 | 2.22 | 2 | 0.02 | 2.94 | 0.37 | 17.94 | 156 | 18 (11.54%) | 3 (16.7%) | 15 (83.3%) | 47 |

NOTE
1. In all the cases pipe diameter is 38 mm

TABLE 3
Comparison of efficiency of different cavitational chamber design to generate cavitation

| | Venturi | NC_Venturi | ven_step4 | Step2 | Ori_ven | Step4 | Ven_ori | Orifice |
|---|---|---|---|---|---|---|---|---|
| | (1a) | (1b) | (2) | (3) | (4) | (5) | (6) | (7) |
| Active (%) | 30 | 40 | 26 | 12 | 29.6 | 19 | 2.5 | 2.19 |
| Transient (%) | 96 | 75 | 94 | 82 | 95 | 98.5 | 50.00 | 56.67 |
| ΔP (atm) | 0.48 | 1.12 | 0.90 | 1.22 | 1.55 | 1.66 | 2.13 | 3.15 |
| Active/ΔP | 62.5 | 35.7 | 28.9 | 9.8 | 19.1 | 11.4 | 1.2 | 0.7 |
| (%/atm) | | | | | | | | |
| Transient/ΔP | 200.0 | 67.0 | 104.4 | 67.2 | 61.3 | 59.3 | 23.5 | 18.0 |
| (%/atm) | | | | | | | | |

TABLE 4
Bacterial analysis in cooling tower water (Example 4)

| Number of | Bacterial count |
|---|---|
| Days | (CFU/ml) |
| 0 | 100000 |
| 6 | 100 |
| 13 | 0 |
| 20 | 0 |

1-19. (canceled)

20. Hydrodynamic cavitation reactors selected from ‘Venturi’, ‘Ven_step4’, ‘Stepped2’, ‘Ori_Ven’, “Ori_Ven”, “Stepped 4”, “Ven_Ori”, Orifice”, “NC_Ven” and combinations thereof, said cavitation reactors comprising cavity generator, flow modulator and/or the turbulence modulator and capable of achieving cavitating conditions in aqueous and non-aqueous media for intensification of the physical and chemical processes, and having cavitation number selected from the range:
0.5 to 1.0 for stable cavitation for ‘Ven_ori’ and ‘Orifice’,
0.22 to 0.5 for transient cavitation for ‘Venturi’, ‘NC_ven’, ‘Venstep4’, ‘Stepped2’, ‘Ori_Ven’, ‘Stepped4’,
0.22 to 0.5 for simultaneous stable and transient cavitation for ‘Ven_ori’ and ‘Orifice’,

21. The Hydrodynamic cavitation reactors as claimed in claim 20 wherein
‘Venturi’ comprises:
a cavity generator which is a portion or whole of minimum cross-sectional area in the cavitation reactor of circular or non circular shape which maximizes the value of perimeter of holes to flow area of holes (α),
a flow modulator, which is a smooth converging section with an overall average angle of 52-56° upstream of the minimum cross sectional area names as the cavity generator and a smooth diverging section with an overall average angle of 20-25° downstream of cavity generator.
‘Ven_step4’ comprises:
a cavity generator which is a portion or whole of minimum cross-sectional area in the cavitation reactor of circular or non circular shape which maximizes the value of perimeter of holes to flow area of holes (α),
turbulence modulator that is downstream of the said cavity generator having multiple sections of length (width) equal to maximum dimension of the cavity generator arranged along the longer axis parallel to the flow with increasing flow area having again an overall average angle 20-25° downstream and held together forming a conduit,
flow modulator that is a smooth converging section with an overall average angle of 52-56° upstream of the cavity generator.
‘Stepped2’ comprises:
a cavity generator which is a portion or whole of minimum cross-sectional area in the cavitation reactor of circular or non circular shape which maximizes the value of perimeter of holes to flow area of holes (α),
turbulence modulator downstream and upstream of the said cavity generator which has sections of length (width) equal to half of the maximum dimension of cavity generator arranged along the longer axis parallel to the flow and held together forming a conduit of increasing flow area having again an overall average angle of 52-56° upstream and 20 -25° downstream;
‘Ori_Ven’, comprises:
a cavity generator which is a portion or hole of minimum cross-sectional area in the cavitation reactor of circular or non circular shape which maximizes the value of the value of perimeter of holes to flow area of holes (α),
flow modulator which is a smooth diverging section with an overall average angle of 20-25° downstream of the cavity generator;
‘Stepped4’ comprises:
a cavity generator which is a portion or whole of minimum cross-sectional area in the cavitation reactor of circular or non circular shape which maximizes the value of perimeter of holes to flow area of holes (α),
turbulence modulator downstream and upstream of the said cavity generator which has sections of length (width) equal to the maximum dimension of cavity generator arranged along the longer axis parallel to the flow and held together forming a conduit of increasing flow area having again an overall average angle of 52-56° upstream and 20 -25° downstream;
‘Ven_Ori’ comprises:
a cavity generator which is portion of minimum cross-sectional area in the cavitation reactor of any shape which maximizes the value of perimeter of holes to flow area of holes (α),
flow modulator which is a smooth converging section with an angle of 52-56° to the upstream of cavity generator;
‘Orifice’ comprises:
a cavity generator which is a portion or whole of minimum cross-sectional area in the cavitation reactor of circular or non circular shape which maximizes the value of perimeter of holes to flow area of holes (a);
‘NC_Ven’ comprises:
a cavity generator which is a portion or whole of minimum cross-sectional area in the cavitation reactor of non circular shape which maximizes the value of perimeter of holes to flow area of holes (α),
flow modulator, which is a smooth converging section with an overall average angle of 52-56° upstream of cavity generator and a smooth diverging section with an average overall angle of 20-25° downstream of cavity generator; maintaining the same or different yet a non-circular shape downstream of the said cavity generator.

22. A cavitation reactor as claimed in claim 20 for microbial cell disruption in heterogeneous system, to operate in stable and transient cavitation wherein the cavitation number is selected from 0.22 to 0.5 preferably 0.28 for a flowrate of 6.73×10⁻⁴ m³/s , wherein the area of holes in orifice is 2.55×10⁻⁵ m² corresponding to a single hole of diameter 5.70 mm, wherein the smallest hole diameter is chosen to maximize the value of a but to a limiting value when hole diameter is 1 mm, thereby amounting to 33 holes to achieve the required total flow area, and active cavitation of 39%, out of which the extent of stable cavitation is 46% resulting in 86% disruption of cells takes place.

23. A cavitation reactor as claimed in claim 20 for Rhodamine degradation to operate in stable cavitation wherein the cavitation number is selected from 0.5 to 1.0 preferably 0.78 to achieve the highest stable cavitation for flowrate of 4.08×10⁻⁴ m³/s wherein area of holes in orifice is 2.59×10⁻⁵ m², corresponding to a single hole of diameter 5.7 mm, wherein the smallest hole diameter is chosen to maximize the value of a but to a limiting value when hole diameter is 1 mm, thereby amounting to 33 holes to achieve the total flow area and stable cavitation of 95% resulting in 17% degradation of Rhodamine.

24. A cavitation reactor as claimed in claim 20 for Toluene oxidation in a heterogeneous liquid-liquid system, to operate in maximized stable cavitation wherein the cavitation number is selected from 0.5 to 1.0 preferably cavitation number of 0.78, more preferably cavitation number of 0.5 for maximized percentage of active cavitation for flowrate of 22.2×10⁻⁴ m³/s wherein the area of holes in orifice is 11.3×10⁻⁵ m² which corresponds to a single hole of diameter 12 mm, wherein optionally the smallest diameter of hole is chosen to maximize the value of a but to a limiting value when diameter of hole is 1 mm or at least 50 times the size of largest rigid/semi rigid particles, resulting in a minimum diameter of hole to ˜2.51 mm thereby amounting to orifice plate with 3 mm diameter of 16 holes to achieve. Stable cavitation of 90.3% resulting in 53% oxidation of toluene or at cavitation number of 0.4 resulting in stable cavitation of 80% to achieve 54% oxidation of toluene.

25. A cavitation reactor as claimed in claim 20 for eliminating biofouling in heterogeneous system, to operate in stable and transient cavitation wherein the cavitation number is selected from 0.5 to 1 preferably 0.8 for a flowrate of 3.14×10⁻² m³/s, wherein the area of cavity generator in venturi is 12.57×10⁻⁴ m² corresponding to a cavity generator of diameter 40 mm, and active cavitation of 26%, out of which the extent of transient cavitation is 10% resulting in 100% decrease in bacterial count.

26. A cavitation reactor as claimed in claim 20 for Esterification of C₈/C₁₀ fatty acids in a heterogeneous liquid-liquid system, to operate in maximized stable cavitation mode wherein the cavitation number is selected from 0.5 to 1.0 preferably cavitation number of 0.78, for maximizing percentage of active cavitation for flowrate of 22.2×10⁻m³/s wherein the area of holes in orifice is 11.3×10 m² which corresponds to a single hole of diameter 12 mm, wherein optionally the smallest diameter of hole is chosen to maximize the value of a but to a limiting value when diameter of hole is 1 mm or at least 50 times the size of largest rigid/semi rigid particles, resulting in a minimum diameter of hole to ˜2.51 mm thereby amounting to orifice plate with 3 mm diameter of 16 holes to achieve stable cavitation of 90.3% resulting in 90% esterification of C₈/C₁₀ fatty acid in 210 mins at a cavitation number of 0.78.

27. A cavitation reactor as claimed in claim 20 for the release of soluble carbon from biomass disruption in heterogeneous system, to operate in transient cavitation wherein the cavitation number is selected from 0.22 to 0.5 preferably 0.5 for venturi with a flowrate of 2.23×10⁻⁴ m³/s, wherein the area of cavity generator in venturi is 1.13×10⁻⁵ m² corresponding to a cavity generator of diameter 4 mm (˜3.8 mm), and active cavitation of 30%, out of which the extent of transient cavitation is 96% resulting in release of 2000 ppm of soluble carbon from the disrupted biomass.

28. A method of tailoring hydrodynamic cavitation reactors to achieve cavitating conditions in aqueous and non-aqueous media using regime maps correlating maximum velocity of fluid or slurry through the cavitation number and percentage of active and/or transient/stable cavitation (FIGS. 1, 2, 4), for intensification of the physical and chemical processes comprising steps of:
Selecting from stable and/or transient cavitation necessary for the targeted physical and/or chemical transformation respectively wherein,
the transient cavitation is selected for chemical transformation in homogenous system;
stable cavitation is selected for chemical transformation in heterogeneous and physical transformations in homogenous system;
stable and transient both are selected for physical transformation in heterogeneous system;
Selecting the cavitation number from a range for the chosen physical or chemical transformation in the first step based on following criteria;
0.5 to 1.0 for stable cavitation for ‘Ven_ori’ and ‘Orifice’,
0.22 to 0.5 for transient cavitation for ‘Venturi’, ‘NC_ven’, ‘Ven_step4’, ‘Stepped2’, ‘Ori_Ven’, ‘Stepped4’,
0.22 to 0.5 for simultaneous stable and transient cavitation for ‘Ven_ori’ and ‘Orifice’.
>Selecting geometry of cavitation reactor from a regime map to maximize the active cavitation for the selected type of cavitation for the selected Cavitation number;
Determining the area of cavity generator within the said selected geometry and the said cavitation number for the volumetric flow rate to be processed using equation 3:
$$Area = \frac{Flow rate}{\sqrt{\frac{{P}_{2} - {P}_{V}}{1 / 2 ρ \cdot {C}_{V}}}} \qquad ( 3 )$$
wherein, Area is area of cavity generator (m²), Flowrate is volumetric flow rate (m³/s), P₂ is pressure downstream to the cavity generator (Pa), Pᵥ is the Vapor pressure of the liquid to be processed for the selected transformation at the operating temperature (Pa), ρ is the density of liquid (kg/m³) and C_{V} is the selected cavitation number;
wherein optionally
when the selected type of geometry of cavitation reactor is an orifice, optimization to maximize active cavitation is done by selecting multiple hole of smallest size such that a, which is ratio of perimeter of holes to flow area of holes, is maximized and sum of the flow area of multiple holes equals the said Area, such that the smallest size of hole is at least 50 times larger than the largest rigid/semi rigid particles in the heterogeneous phase, wherein the smallest size of hole is limited to 1 mm;
if in a Liquid-Liquid heterogeneous system involving emulsification step that has a preceding chemical transformation, an additional criterion of Weber number=4.7 is chosen;
wherein, Weber number (We) is defined as the ratio of inertial forces responsible for breakup to interfacial forces resisting the breakup
$$We = \frac{{d}_{E} {v}^{′2} ρ}{σ}$$
Wherein, d_{E} is the size of emulsion, v′ is the turbulent fluctuating velocity, ρ is the density of liquid and σ is interfacial surface tension;
if the selected type of said geometry of cavitation reactor is a multiple orifice, the spacing of the holes is obtained from
dₛ=dₕ+4×10⁻⁴V_{J}
where, dₛ is the spacing between the holes (m); dₕ is minimum dimension of the hole (m) and V_{J} is the velocity of the liquid at cavity generator (m/s).

29. A method of tailoring hydrodynamic cavitation reactors to achieve cavitating conditions in aqueous and non-aqueous media for intensification of the physical and chemical processes as claimed in claim 28 wherein cavitation reactor is a ‘Venturi’ comprising:
a cavity generator which is a portion or whole of minimum cross-sectional area in the cavitation reactor of circular or non circular shape which maximizes the value of perimeter of holes to flow area of holes (a),
a flow modulator, which is a smooth converging section with an overall average angle of 52-56° upstream of the minimum cross sectional area names as the cavity generator and a smooth diverging section with an overall average angle of 20-25° downstream of cavity generator.

30. A method of tailoring hydrodynamic cavitation reactors to achieve cavitating conditions in aqueous and non-aqueous media for intensification of the physical and chemical processes as claimed in claim 28 wherein the cavitation reactor is an ‘Orifice’ comprising
a. a cavity generator which is a portion or whole of minimum cross-sectional area in the cavitation reactor of circular or non circular shape which maximizes the value of perimeter of holes to flow area of holes α.

31. A method of tailoring hydrodynamic cavitation reactors to achieve cavitating conditions in aqueous and non-aqueous media for intensification of the physical and chemical processes as claimed in claim 28
wherein the cavitation reactor is a ‘Ven_step4’ comprising:
a. a cavity generator which is a portion or whole of minimum cross-sectional area in the cavitation reactor of circular or non circular shape which maximizes the value of perimeter of holes to flow area of holes (α),
b. turbulence modulator that is downstream of the said cavity generator having multiple sections of length (width) equal to maximum dimension of the cavity generator arranged along the longer axis parallel to the flow with increasing flow area having again an overall average angle 20 -25° downstream and held together forming a conduit,
c. flow modulator that is a smooth converging section with an overall average angle of 52-56° upstream of the cavity generator.
or
wherein the cavitation reactor is a ‘Stepped2’ comprising:
d. a cavity generator which is a portion or whole of minimum cross-sectional area in the cavitation reactor of circular or non circular shape which maximizes the value of perimeter of holes to flow area of holes (α),
e. turbulence modulator downstream and upstream of the said cavity generator which has sections of length (width) equal to half of the maximum dimension of cavity generator arranged along the longer axis parallel to the flow and held together forming a conduit of increasing flow area having again an overall average angle of 52-56° upstream and 20 -25° downstream.
or
wherein the cavitation reactor is a ‘Ori_Ven’, comprises
f. a cavity generator which is a portion or hole of minimum cross-sectional area in the cavitation reactor of circular or non circular shape which maximizes the value of perimeter of holes to flow area of holes (α),
g. flow modulator which is a smooth diverging section with an overall average angle of 20-25° downstream of the cavity generator.
or
wherein the cavitation reactor is a “Stepped4’ comprising
h. a cavity generator which is a portion or whole of minimum cross-sectional area in the cavitation reactor of circular or non circular shape which maximizes the value of perimeter of holes to flow area of holes (α),
i. turbulence modulator downstream and upstream of the said cavity generator which has sections of length (width) equal to the maximum dimension of cavity generator arranged along the longer axis parallel to the flow and held together forming a conduit of increasing flow area having again an overall average angle of 52-56° upstream and 20 -25° downstream;
or
wherein the cavitation reactor is a ‘Ven_Ori’ comprising
j. a cavity generator which is portion of minimum cross-sectional area in the cavitation reactor of any shape which maximizes the value of perimeter of holes to flow area of holes (α),
k. flow modulator which is a smooth converging section with an angle of 52-56° to the upstream of cavity generator.
or
wherein the cavitation reactor is a ‘NC_Ven’ comprising
l. a cavity generator which is a portion or whole of minimum cross-sectional area in the cavitation reactor of non circular shape which maximizes the value of perimeter of holes to flow area of holes (α),
m. flow modulator, which is a smooth converging section with an overall average angle of 52-56° upstream of cavity generator and a smooth diverging section with an average overall angle of 20-25° downstream of cavity generator; maintaining the same or different yet a non-circular shape downstream of the said cavity generator.

32. Regime maps as claimed in claim 28 correlating maximum velocity of fluid or slurry through the cavitation reactor, cavitation number and percentage of active, transient and stable cavitation as in FIGS. 1, 2 & 4 is obtained by a process comprising steps:
establishing, over the cavitation reactor, the material continuity and the balance of momentum, turbulent kinetic energy and turbulent energy dissipation rate using appropriate equation consisting of fundamental variables like (P) pressure over the liquid, (u) velocity component in x direction, (v) velocity component in y direction, (w) velocity component in z direction, (k) turbulent kinetic energy, (□) turbulent energy dissipation rate, (ρ) liquid density, (σ) liquid phase surface and interfacial tension, (μ) liquid viscosity;
wherein, continuity equation is
$$\frac{\partial ρ}{\partial t} + ∇ \cdot ( ρ \overline{u} ) = 0$$
wherein, momentum balance equation is
$$\frac{\partial }{\partial t} ( ρ \overline{u} ) + ∇ \cdot ( ρ \overline{u} \overline{u} ) = - ∇ P - ∇ \cdot ( ρ {\overline{u}}^{′} {\overline{u}}^{′} ) + μ {∇}^{2} {\overline{u}}_{i} + ρ \overline{g}$$
wherein, turbulent kinetic energy equation is
$$\frac{\partial }{\partial t} ( ρ k ) + \frac{\partial }{\partial {x}_{i}} ( ρ k {u}_{i} ) = \frac{\partial }{\partial {x}_{j}} [ ( μ + 0.09 ρ \frac{{k}^{2}}{ɛ} ) \frac{\partial k}{\partial {x}_{j}} ] - ( ρ {\overline{u}}_{i} {\overline{u}}_{j} \frac{\partial {u}_{j}}{\partial {x}_{i}} ) - ρɛ$$
wherein, turbulent energy dissipation rate equation is
$$\frac{\partial }{\partial t} ( ρ ɛ ) + \frac{\partial }{\partial {x}_{i}} ( ρɛ {u}_{i} ) = \frac{\partial }{\partial {x}_{j}} [ ( μ + 0.069 ρ \frac{{k}^{2}}{ɛ} ) \frac{\partial ɛ}{\partial {x}_{j}} ] + 1.44 \frac{ɛ}{k} ( ρ {\overline{u}}_{i} {\overline{u}}_{j} \frac{\partial {u}_{j}}{\partial {x}_{i}} ) - 1.92 ρ \frac{{ɛ}^{2}}{k}$$
wherein, the above equations are solved numerically to obtain P, k & □;
Obtaining the ‘n’ number of likely paths taken by the cavities through the cavitation reactor
wherein, n is any integer, significantly greater than 100;
wherein paths taken by cavity is obtained from Lagrangian equation
$$\frac{\partial {u}_{P}}{\partial t} = {F}_{D} ( u - {u}_{P} ) + \frac{{g}_{x} ( {ρ}_{P} - ρ )}{{ρ}_{P}} \qquad ( L )$$
Wherein, U_{P} is the cavity velocity, F_{D}(μ-μ_{P}) is drag force per unit mass of cavity, ρ_{P} is the density of cavity, t is time, gₓ is gravitational acceleration in x direction (Table 1);
wherein, Lagrangian equation (L) is solved numerically to obtain the time dependent co-ordinates of the cavity;
wherein, P, k and [symbol]
are obtained from the solution of balances at these co-ordinates obtained from Lagrangian equation (L);
obtaining the value of pressure amplitude (Pₐₘₚ), pressure frequency (f) and Instantaneous pressure sensed by the cavity (P_{∞}) from relations
$${P}_{amp} = 1 / 3 ρ k ; f = \frac{ɛ}{k} ; {P}_{∞} ( t ) = P - {P}_{amp} sin ( 2 π ft ) ;$$
obtaining the cavity dynamics (cavity radius as a function of time) from cavity dynamics models using the above data of P_{∞}, Pₐₘₚ, f;
wherein, the cavity dynamics models are generally known as Rayleigh-Plesset family of equations e.g.
$$R ( \frac{{d}^{2} R}{d {t}^{2}} ) + \frac{3}{2} {( \frac{d R}{d t} )}^{2} = \frac{1}{{ρ}_{l}} [ {P}_{B} - \frac{4 μ}{R} ( \frac{d R}{d t} ) - \frac{2 σ}{R} - {P}_{∞} ]$$
wherein, t is time, R is radius of cavity at any instant, σ is liquid surface tension, μ is liquid viscosity, P_{B} is pressure inside the bubble;
Categorizing the cavities as active, stable and transient cavitation using the following criteria;
wherein, a cavity is active if pressure inside the cavity is more than 10 times the pressure at the inlet of cavitation reactor,
wherein, an active cavity is a stable cavity if final pressure is not equal to maximum pressure inside the cavity during its lifetime,
wherein, an active cavity is a transient cavity if final oscillating pressure is equal to the maximum pressure inside the cavity,
Calculating, for a given velocity, cavitation number, selected geometry (shape and size) of the cavitation reactor,
The percentage of active cavitation as number of active cavities/total number of cavities×100,
The percentage of stable cavitation as number of stable cavities/total number of active cavities×100,
The percentage of transient cavitation as number of transient cavities/total number of active cavities×100.

33. Method as claimed in claim 28 wherein the liquids are selected from those having density: 850-1500 kg/m³, viscosity: 1-100 cP, surface tension: 0.01-0.075 N/m, and liquid vapor pressure: 300-101325 Pa.
